(19) United States
(12) Patent Application Publication
(10) Pub. No.: US 2015/0361407 A1
(43) Pub. Date: Dec. 17, 2015

(54) ENZYME PRODUCTION COMPOSITIONS AND METHODS

(71) Applicant: CODEXIS, INC., Redwood City, CA (US)

(72) Inventors: Lorand Szabo, Budapest (HU); Jeno Szilagyi, Debrecen (HU); Kata Zsuzsanna Hetenyi, Soponya (HU); Dipnath Baidyaroy, Fremont, CA (US)

(21) Appl. No.: 14/758,841

(22) PCT Filed: Jan. 9, 2014
(86) PCT No.: PCT/US14/10814

§ 371 Date: Jul. 1, 2015

Related U.S. Application Data
(60) Provisional application No. 61/751,492, filed on Jan. 11, 2013.

(51) Int. Cl. C12N 9/24; C12P 19/14; C12P 19/02

(52) U.S. Cl. CPC C12N 9/2402; C12P 19/02; C12P 19/14; C12Y 101/03004; C12Y 302/01021; C12Y 302/01091; C12Y 302/01008; C12Y 503/01005

(57) ABSTRACT

The present invention provides compositions and methods for the production of enzymes. Interest has arisen in fermentation of carbohydrate-rich biomass to provide alternatives to petrochemical sources for fuels and organic chemical precursors. “First generation” bioethanol production from carbohydrate sources (e.g., sugar cane, corn, wheat, etc.) have proven to be marginally economically viable on a production scale.

The present application claims priority to U.S. Provisional Application Ser. No. 61/751,492, filed Jan. 11, 2013, incorporated herein by reference in its entirety for all purposes.

### FIELD OF THE INVENTION

The present invention provides compositions and methods for the production of enzymes.

### BACKGROUND

Interest has arisen in fermentation of carbohydrate-rich biomass to provide alternatives to petrochemical sources for fuels and organic chemical precursors. “First generation” bioethanol production from carbohydrate sources (e.g., sugar cane, corn, wheat, etc.) have proven to be marginally economically viable on a production scale. “Second generation” bioethanol produced using lignocellulosic feedstocks has faced significant obstacles to commercial viability. Bioethanol is currently produced by the fermentation of hexose sugars that are obtained from carbon feedstocks. There is great interest in using lignocellulosic feedstocks where the plant cellulose is broken down to sugars and subsequently converted to ethanol. Lignocellulosic biomass is primarily composed of cellulose, hemicelluloses, and lignin. Cellulose and hemicellulose can be hydrolyzed in a saccharification process to sugars that can be subsequently converted to ethanol via fermentation. The major fermentable sugars from lignocelluloses are glucose and xylose. For economical ethanol yields, a process that can effectively convert all the major sugars present in cellulosic feedstock would be highly desirable.

### SUMMARY OF THE INVENTION

The present invention provides compositions and methods for the production of enzymes.

In some embodiments, the present invention provides methods for production of at least one enzyme from a fungus of the genus Myceliophthora comprising providing a fungal cell of the genus Myceliophthora or a taxonomically equivalent genus capable of producing at least one enzyme and a culture medium comprising low cellulose, contacting the fungal cell and the culture medium under conditions such that the fungal cell produces at least one enzyme. In some embodiments, the medium comprising low cellulose comprises less that about 50 g/L cellulose in the starting culture, while in some additional embodiments, the medium comprise less than about 45 g/L cellulose, less than about 40 g/L, less than about 35 g/L, less than about 30 g/L, less than about 25 g/L, less than about 20 g/L, less than about 15 g/L, less than about 10 g/L, less than about 5 g/L, or less than less than about 2.5 g/L. The present invention also provides methods for production of enzymes from a fungus of the genus Myceliophthora comprising providing a fungal cell of the genus Myceliophthora or a taxonomically equivalent genus capable of producing at least one enzyme and a culture medium comprising no cellulose, contacting the fungal cell and the culture medium under conditions such that the fungal cell produces at least one enzyme. In some embodiments, the culture medium comprises no added cellulose in that no cellulose-containing supplement is added to the medium, but a substrate within the culture medium (e.g., biomass) comprises cellulose. In some embodiments, the medium comprising no cellulose comprises no detectable levels of cellulose, using standard methods known in the art to identify and/or quantitate cellulose. In some embodiments, the contacting occurs at a pH of about 4 to about 10, about 5 to about 9, about 6 to about 8, about 5 to about 7, or about 4 to about 9. In some further embodiments, the pH is between about 5 and about 7. In some embodiments, the pH is about 6. Indeed, it not intended that the present invention be limited to any particular pH, as any suitable pH finds use with the present invention. In some embodiments, the contacting comprises a batch, fed-batch, continuous, and/or repeated fed-batch culturing method. In some further embodiments, the methods are batch, fed-batch, and/or repeated fed-batch culturing methods. In some additional embodiments, the batch, fed-batch, and/or repeated fed-batch culturing methods comprise adding at least one feed solution to the culture medium. In still some further embodiments, the feed solution comprises at least one carbon source and/or at least one nitrogen source, including, but not limited to the compounds listed in Table 2.6, herein. In some embodiments, the carbon source is selected from monosaccharides, disaccharides, polysaccharides, alcohols, molasses, polyols, glycerol, and sucrose. However, it is not intended that the present invention be limited to any particular carbon and/or nitrogen source in the feed solution. In some embodiments, the feed solution comprises glucose, while in some alternative embodiments, the feed solution does not comprise glucose. In some additional embodiments, the feed solution comprises glucose and at least one supplemental composition, including but limited to ((NH₄)₂SO₄), salts, micro elements, and/or any additional suitable compositions, including but not limited to those listed in Tables 2-4, 2-5, and/or 2-6. In some further embodiments, the fungal cell is contacted with at least one adjunct composition. In some embodiments, the adjunct composition is selected from reducing agents, gallic acid, surfactants, and divalent metal cations, vitamins, and/or polyethylene glycol. In some further embodiments, the adjunct composition comprises at least one divalent metal cation. In some embodiments, the divalent metal cation comprises copper. However, it is not intended that the present invention be limited to any particular adjunct composition, as any suitable composition for the desired purpose finds use in the present invention. In some embodiments, the fungal cell produces at total protein levels of at least about 2.5 g/L, at least about 5 g/L, at least about 10 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, at least about 60 g/L, at least about 65 g/L, at least about 70 g/L, at least about 75 g/L, at least about 80 g/L, a at least about 85 g/L, at least about 90 g/L, at least about 95 g/L, or at least about 100 g/L. In some further embodiments, the total protein produced is at least about 25 g/L, at least about 30 g/L, at least about 35 g/L, at least about 40 g/L, at least about 45 g/L, at least about 50 g/L, at least about 55 g/L, at least about 60 g/L, at least about 65 g/L, at least about 70 g/L, at least about 75 g/L, at least about 80 g/L, at least about 85 g/L, at least about 90 g/L, at least about 95 g/L, or at least about 100 g/L. In some additional embodiments, the methods are conducted in a reaction volume of at least about 15 L, at least about 20 L, at least about 25 L, at least about 30 L, at least about 35 L, at least about 40 L, at least about 45 L, at least about 50 L, at least about 55 L, at least about 60 L, at least about 65 L, at least about 70 L, at least about 75 L, at least about 80 L, at least about 85 L, at least about 90 L, at least about 95 L, at least about 100 L, at least about 150 L, at least about 200 L, at least about 250 L, at least about 300 L, at least about 350 L, at least about 400 L, at least about 450 L, at least about 500 L, at least about 550 L, at least about 600 L, at least about 650 L, at least about 700 L, at least about 750 L, at least about 800 L, at least about 850 L, at least about 900 L, at least about 950 L, at least about 1000 L, at least about 1500 L, at least about 2000 L, at least about 2500 L, at least about 3000 L, at least about 3500 L, at least about 4000 L, at least about 4500 L, at least about 5000 L, at least about 5500 L, at least about 6000 L, at least about 6500 L, at least about 7000 L, at least about 7500 L, at least about 8000 L, at least about 8500 L, at least about 9000 L, at least about 9500 L, at least about 10,000 L, at least about 10,500 L, at least about 20,000 L, at least about 25,000 L, at least about 30,000 L, at least about 35,000 L, at least about 40,000 L, at least about 45,000 L, at least about 50,000 L, at least about 55,000 L, at least about 60,000 L, at least about 65,000 L, at least about 70,000 L, at least about 75,000 L, at least about 80,000 L, at least about 85,000 L, at least about 90,000 L, or at least about 100,000 L. In some alternative embodiments, the methods are conducted in a reaction volume of at least about 100,000 L, at least about 150,000 L, at least about 200,000 L, at least about 250,000 L, at least about 300,000 L, at least about 350,000 L, at least about 400,000 L, at least about 450,000 L, or at least about 500,000 L. In some further embodiments, the methods produce at least one enzyme, wherein at least one enzyme is a cellulase. In some further embodiments, the methods produce at least one enzyme selected from CBHs, EGs, BGLs, GH61 enzymes, xylanases, glucanases, pectinases, amylases, glucoamylases, lipases, proteases, esterases, glucose isomerases, glucose oxidases, phytases, etc. Indeed, it is not intended that the present invention be limited to the production of any particular enzyme(s), as the methods find use in the production of numerous enzymes of interest. In some additional embodiments, the fungal cell produces at least two cellulolytic enzymes. In some embodiments, the fungal cell produces at least one cellulase and at least one additional enzyme. In some further embodiments, the fungal cell produces at least two cellulases and at least one additional enzyme. In some embodiments, the additional enzyme is selected from CBHs, EGs, BGLs, GH61 enzymes, xylanases, glucanases, pectinases, amylases, glucoamylases, lipases, proteases, esterases, glucose isomerases, glucose oxidases, and phytases, etc. indeed, it is not intended that the present invention be limited to any particular enzyme and/or enzyme class.

In some embodiments of the present invention, at least one cellulolytic enzyme produced using the methods provided herein comprises an enzyme composition that is contacted with at least one cellulosic substrate under conditions whereby fermentable sugars are produced. In some embodiments, the cellulolytic enzyme is purified prior to contacting with at least one cellulosic substrate, while in some alternative embodiments, the cellulolytic enzyme is not purified prior to contacting with at least one cellulosic substrate. In some further embodiments, at least one cellulolytic enzyme is present in a whole broth preparation that is contacted with at least one cellulosic substrate. In still some additional embodiments, at least one cellulolytic enzyme is combined with at least one purified enzyme composition. In some further embodiments, at least one purified enzyme is a purified cellulolytic enzyme. In some additional embodiments, the purified enzyme composition comprises at least one CBH, at least one EG, at least one BGL, at least one GH61 enzyme, at least one xylanase, at least one glucanase, at least one pectinase, at least one amylase, at least one glucoamylase, at least one lipase, at least one protease, at least one esterase, at least one glucose isomerase, at least one glucose oxidase, and/or at least one phytase. In some embodiments, the methods further comprise pretreating the cellulosic substrate prior to contacting the substrate with the enzyme composition, while in some alternative embodiments, the enzyme composition is added concurrently with pretreating. In some embodiments, the cellulosic substrate comprises wheat grass, wheat straw, barley straw, sorghum, rice grass, sugarcane straw, bagasse, switchgrass, corn stover, corn fiber, grains, or any combination thereof. In some embodiments, the fermentable sugars comprise glucose and/or xylose. In some further embodiments, the methods further comprise recovering the fermentable sugars. In some embodiments, the conditions comprise using continuous, batch, and/or fed-batch culturing conditions. In some embodiments, the methods are batch process, while in some other embodiments, the methods are continuous processes, fed-batch processes, and/or repeated fed-batch processes. In some additional embodiments, the methods comprise any combination of batch, continuous, and/or fed-batch processes conducted in any order. In some further embodiments, the methods are conducted in vessels comprising reaction volumes of at least about 15 L, at least about 20 L, at least about 25 L, at least about 30 L, at least about 35 L, at least about 40 L, at least about 45 L, at least about 50 L, at least about 55 L, at least about 60 L, at least about 65 L, at least about 70 L, at least about 75 L, at least about 80 L, at least about 85 L, at least about 90 L, at least about 95 L, at least about 100 L, at least about 150 L, at least about 200 L, at least about 250 L, at least about 300 L, at least about 350 L, at least about 400 L, at least about 450 L, at least about 500 L, at least about 550 L, at least about 600 L, at least about 650 L, at least about 700 L, at least about 750 L, at least about 800 L, at least about 850 L, at least about 900 L, at least about 950 L, at least about 1000 L, at least about 1500 L, at least about 2000 L, at least about 2500 L, at least about 3000 L, at least about 3500 L, at least about 4000 L, at least about 4500 L, at least about 5000 L, at least about 5500 L, at least about 6000 L, at least about 6500 L, at least about 7000 L, at least about 7500 L, at least about 8000 L, at least about 8500 L, at least about 9000 L, at least about 9500 L, at least about 10,000 L, at least about 10,500 L, at least about 20,000 L, at least about 25,000 L, at least about 30,000 L, at least about 35,000 L, at least about 40,000 L, at least about 45,000 L, at least about 50,000 L, at least about 55,000 L, at least about 60,000 L, at least about 65,000 L, at least about 70,000 L, at least about 75,000 L, at least about 80,000 L, at least about 85,000 L, at least about 90,000 L, at least about 100,000 L, at least about 150,000 L, at least about 200,000 L, at least about 250,000 L, at least about 300,000 L, at least about 350,000 L, at least about 400,000 L, at least about 450,000 L, at least about 500,000 L, at least about 550,000 L, at least about 600,000 L, at least about 650,000 L, at least about 700,000 L, at least about 750,000 L, at least about 800,000 L, at least about 850,000 L, at least about 900,000 L, at least about 950,000 L, at least about 1,000,000 L, or larger.

The present invention also provides methods for producing at least one end product from at least one cellulosic substrate, comprising: a) providing at least one cellulosic substrate and at least one enzyme composition as provided herein; b) contacting the cellulosic substrate with the enzyme composition under conditions whereby fermentable sugars are produced from the cellulosic substrate in a saccharification reaction; and c) contacting the fermentable sugars with a microorganism under fermentation conditions such that at least one end product is produced. In some embodiments, the methods comprise simultaneous saccharification and fermentation reactions (SSF), while in some alternative embodiments, the methods, saccharification of the cellulosic substrate and fermentation are conducted in separate reactions (SHF). In some additional embodiments, the enzyme composition is produced simultaneously with saccharification and/or fermentation reaction(s). In some further embodiments, the methods further comprise at least one adjunct composition in the saccharification reaction. In some embodiments, the adjunct composition is selected from at least one divalent metal cation, gallic acid, and/or at least one surfactant. In still some additional embodiments, the divalent metal cation comprises copper. In some further embodiments, the adjunct composition comprises gallic acid. In some embodiments, the surfactant is selected from TWEEN®-20 non-ionic detergent and polyethylene glycol. In some additional embodiments, the methods are conducted at about pH 4.0 to about pH 7.0. In some additional embodiments, the methods are conducted at about pH 5.0, while in some alternative embodiments, the methods are conducted at about pH 6.0. In some embodiments, the methods further comprise recovering at least one end product. In some additional embodiments, the end product comprises at least one fermentation end product. In some further embodiments, the fermentation end product is selected from alcohols, fatty acids, lactic acid, acetic acid, 3-hydroxypropionic acid, acrylic acid, succinic acid, citric acid, malic acid, fumaric acid, an amino acid, 1,3-propanediol, ethylene, glycerol, fatty alcohols, butadiene, and beta-lactams. In some embodiments, the fermentation end product comprises at least one alcohol selected from ethanol and butanol. In some additional embodiments, the alcohol is ethanol. In some further embodiments, the microorganism is a yeast. In some embodiments, the yeast is Saccharomyces. In yet some additional embodiments, the methods further comprise recovering at least one fermentation end product.

### DESCRIPTION OF THE INVENTION

The present invention provides compositions and methods for the production of enzymes.

All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, some suitable methods and materials are described. Indeed, it is intended that the present invention not be limited to the particular methodology, protocols, and reagents described herein, as these may vary, depending upon the context in which they are used. The headings provided herein are not limitations of the various aspects or embodiments of the present invention.

Nonetheless, in order to facilitate understanding of the present invention, a number of terms are defined below. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention that can be had by reference to the specification as a whole. Accordingly, the terms defined below are more fully defined by reference to the specification as a whole.

As used herein, the terms “cellulase” and “cellulolytic enzyme” refer to any enzyme that is capable of degrading cellulose. Thus, the term encompasses enzymes capable of hydrolyzing cellulose (β-1,4-glucan and/or β-D-glucosides) to shorter cellulose chains, oligosaccharides, cellobiose and/or glucose. “Cellulases” are divided into three sub-categories of enzymes: 1,4-β-D-glucan glucanohydrolase (“endoglucanase” or “EG”); 1,4-β-D-glucan cellobiohydrolase (“exoglucanase,” “cellobiohydrolase,” or “CBH”); and β-D-glucoside-glucohydrolase (“β-glucosidase,” “cellobiase,” “BG,” or “BGL”). These enzymes act in concert to catalyze the hydrolysis of cellulose-containing substrates. Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble β-1,4-linked dimer of glucose) as well as glucose, cellotriose, and cellotetrose. Beta-glucosidases split the cellobiose and soluble cellodextrins into glucose. Some enzymes (e.g., “celluolytic enhancing,” or “cellulolytic-activity enhancing” enzymes) act to enhance the activity of other cellulases, thereby increasing the breakdown of cellulose (i.e., as compared to the activity of the other cellulases without the presence of the cellulolytic enhancing enzyme(s).

A “hemicellulase” as used herein, refers to a polypeptide that can catalyze hydrolysis of hemicellulose into small polysaccharides such as oligosaccharides, or monomeric saccharides. Hemicelluloses include xylan, glucuonoxylan, arabinoxylan, glucomannan and xyloglucan. Hemicellulases include, for example, the following: endoxylanases, b-xylosidases, a-L-arabinofuranosidases, a-D-glucuronidases, feruloyl esterases, coumnaroyl esterases, a-galactosidases, b-galactosidases, b-mannanases, and b-mannosidases. In some embodiments, the present invention provides enzyme mixtures that comprise EG1b and one or more hemicellulases.

As used herein, “cellulose” refers to compositions comprising β-1,4-glucan.

As used herein, “protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the present invention. Some specific types of proteases include but are not limited to, cysteine proteases including pepsin, papain and serine proteases including chymotrypsins, carboxypeptidases and metalloendopeptidases.

As used herein, “lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

As used herein, the terms “isolated” and “purified” are used to refer to a molecule (e.g., an isolated nucleic acid, polypeptide, etc.) or other component that is removed from at least one other component with which it is naturally associated.

As used herein, “polynucleotide” refers to a polymer of deoxyribonucleotides or ribonucleotides in either single- or double-stranded form, and complements thereof.

The terms “protein” and “polypeptide” are used interchangeably herein to refer to a polymer of amino acid residues.

In addition, the terms “amino acid” “polypeptide,” and “peptide” encompass naturally-occurring and synthetic amino acids, as well as amino acid analogs. Naturally occurring amino acids are those encoded by the genetic code, as well as those amino acids that are later modified (e.g., hydroxyproline, γ-carboxygiutamate, and O-phosphoserine). As used herein, the term “amino acid analogs” refers to compounds that have the same basic chemical structure as a naturally occurring amino acid (i.e., an α-carbon that is bound to a hydrogen, a carboxyl group, an amino group, and an R group, including but not limited to homoserine, norleucine, methionine sulfoxide, and methionine methyl sutfonium). In some embodiments, these analogs have modified R groups (e.g., norleucine) and/or modified peptide backbones, but retain the same basic chemical structure as a naturally occurring amino acid. Amino acids are referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “overexpress” is intended to encompass increasing the expression of a protein to a level greater than the cell normally produces. It is intended that the term encompass overexpression of endogenous, as well as heterologous proteins.

As used herein, the term “recombinant” refers to a polynucleotide or polypeptide that does not naturally occur in a host cell. In some embodiments, recombinant molecules contain two or more naturally-occurring sequences that are linked together in a way that does not occur naturally. In some embodiments, “recombinant cells” express genes that are not found in identical form within the native (i.e., non-recombinant) form of the cell and/or express native genes that are otherwise abnormally over-expressed, under-expressed, and/or not expressed at all due to deliberate human intervention. Recombinant cells contain at least one recombinant polynucleotide or polypeptide. A nucleic acid construct, nucleic acid (e.g., a polynucleotide), polypeptide, or host cell is referred to herein as “recombinant” when it is non-naturally occurring, artificial or engineered. “Recombination,” “recombining” and generating a “recombined” nucleic acid generally encompass the assembly of at least two nucleic acid fragments.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. An “expression vector” has a promoter sequence operably linked to the DNA sequence (e.g., transgene) to drive expression in a host cell, and in some embodiments a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein. In some embodiments, the host cells are prokaryotic or eukaryotic cells that have been transformed or transfected with vectors constructed using recombinant DNA techniques as known in the art. Transformed hosts are capable of either replicating vectors encoding at least one protein of interest and/or expressing the desired protein of interest. In addition, reference to a cell of a particular strain refers to a parental cell of the strain as well as progeny and genetically modified derivatives. Genetically modified derivatives of a parental cell include progeny cells that contain a modified genome or episomal plasmids that confer for example, antibiotic resistance, improved fermentation, etc. In some embodiments, host cells are genetically modified to have characteristics that improve protein secretion, protein stability or other properties desirable for expression and/or secretion of a protein. For example, knockout of Alp1 function results in a cell that is protease deficient. Knockout of pyr5 function results in a cell with a pyrimidine deficient phenotype. In some embodiments, host cells are modified to delete endogenous cellulase protein-encoding sequences or otherwise eliminate expression of one or more endogenous cellulases. In some embodiments, expression of one or more endogenous cellulases is inhibited to increase production of cellulases of interest. Genetic modification can be achieved by any suitable genetic engineering techniques and/or classical microbiological techniques (e.g., chemical or UV mutagenesis and subsequent selection). Using recombinant technology, nucleic acid molecules can be introduced, deleted, inhibited or modified, in a manner that results in increased yields of EG1b within the organism or in the culture. For example, knockout of Alp1 function results in a cell that is protease deficient. Knockout of pyr5 function results in a cell with a pyrimidine deficient phenotype. In some genetic engineering approaches, homologous recombination is used to induce targeted gene modifications by specifically targeting a gene in vivo to suppress expression of the encoded protein. In an alternative approach, siRNA, antisense, and/or ribozyme technology finds use in inhibiting gene expression.

As used herein, the term “introduced” used in the context of inserting a nucleic acid sequence into a cell, means transformation, transduction, conjugation, transfection, and/or any other suitable method(s) known in the art for inserting nucleic acid sequences into host cells. Any suitable means for the introduction of nucleic acid into host cells find use in the present invention.

As used herein, the terms “transformed” and “transformation” used in reference to a cell refer to a cell that has a non-native nucleic acid sequence integrated into its genome or has an episomal plasmid that is maintained through multiple generations.

In some embodiments, the expression vector of the present invention contains one or more selectable markers, which permit easy selection of transformed cells. A “selectable marker” is a gene, the product of which provides for biocide or viral resistance, resistance to antimicrobials or heavy metals, prototrophy to auxotrophy, and the like. Any suitable selectable markers for use in a filamentous fungal host cell find use in the present invention, including, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Additional markers useful in host cells such as Aspergillus, include but are not limited to the amdS and pyrG genes of Aspergillus nidulans or Aspergillus oryzae and the bar gene of Streptomyces hygroscopicus. Suitable markers for yeast host cells include, but are not limited to ADE2, HIS3, LEU2, LYS2, MET3, TRP1, and URA3.

In some embodiments, the engineered host cells (i.e., “recombinant host cells”) of the present invention are cultured in conventional nutrient media modified as appropriate for activating promoters, selecting transformants, or amplifying cellulase polynucleotides. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

As used herein, the terms “culture medium” and “medium formulation” refer to nutritive solutions for the production, maintenance, growth, propagation, and/or expansion of cells (e.g., fungi) in an in vitro environment (e.g., shake flasks, tanks, etc.). Indeed it is intended that any suitable medium will find use in the present invention. Furthermore, in some embodiments, the media comprise cellulose, while in some other embodiments, the media do not comprise cellulose (i.e., measurable concentrations of cellulose). In some additional embodiments, the media comprise carbon sources such as glucose, dextrose, etc. However, it is not intended that the present invention be limited to any specific carbon and/or nitrogen source, as any suitable carbon and/or nitrogen source finds use in the present invention. It is not intended that the present invention be limited to any particular medium, as any suitable medium will find use in the desired setting.

As used herein, the terms “nutrient,” “ingredient,” and “component” are used interchangeably to refer to the constituents that make up a culture medium.

As used herein, the term “basal medium” refers to any culture medium that is capable of supporting the growth of cells, including fungal cells (e.g., M. thermophila).

As used herein, the term “modified basal medium” refers to a basal medium from which at least one standard ingredient, component or nutrient (i.e., at least one ingredient, component or nutrient found in standard basal media known in the art) has been excluded, decreased, or increased. In some embodiments, as determined by context, the term “modified” as used in the context of “modified basal medium” also refers to changes in proportions between the individual components within the basal medium. In some embodiments, a modified basal medium of the present invention comprises a reduced concentration of cellulose as compared to standard fungal media known in the art. In some additional embodiments, the modified basal medium comprises no cellulose (i.e., no cellulose is added to the medium).

As used herein, the terms “adjunct material,” “adjunct composition,” and “adjunct compound” refer to any composition suitable for use in the compositions and/or methods provided herein, including but not limited to cofactors, surfactants, builders, buffers, enzyme stabilizing systems, chelants, dispersants, colorants, preservatives, antioxidants, solubilizing agents, carriers, processing aids, pH control agents, etc. In some embodiments, divalent metal cations are used to supplement saccharification reactions and/or the growth of fungal cells. Any suitable divalent metal cation finds use in the present invention, including but not limited to Cu⁺⁺, Mn⁺⁺, Co⁺⁺, Mg⁺⁺, Ni⁺⁺, Zn⁺⁺, and Ca⁺⁺. In addition, any suitable combination of divalent metal cations finds use in the present invention. Furthermore, divalent metal cations find use from any suitable source.

As used herein, “inoculation medium” refers to the culture media used to produce an aliquot of organisms (i.e., an “inoculum”) for use in inoculating a culture medium (e.g., production medium) to facilitate growth of the organisms and production of desired product(s) (e.g., enzymes).

As used herein, the term “inoculation” refers to the addition of cells (e.g., fungal cells) to begin a culture (e.g., a fungal culture).

As used herein, the terms “culture production medium” and “production medium” refer to culture media designed to be used during the production phase of a culture. In some embodiments, production media are designed for recombinant protein production during fungal growth.

In some embodiments, cells expressing the cellulase(s) of the invention are grown under batch or continuous culture conditions. Combinations and/or variations of unique characteristics of these processes find use in various embodiments of the present invention. Indeed, it is not intended that the present invention be limited to any specific growth protocol and/or method. Classical “batch culturing” involves a closed system, wherein the composition of the medium is set at the beginning of the culture process and is not subject to artificial alternations during the culture process. A variation of the batch system is “fed-batch culturing” which also finds use in the present invention. In this variation, the substrate is added in increments as the culturing process progresses. Fed-batch systems are useful when catabolite repression is likely to inhibit the metabolism of the cells and where it is desirable to have limited amounts of substrate in the medium. Batch and fed-batch cultures are common and well known in the art. In some additional embodiments, “repeated fed-batch” culturing finds use in the present invention. In these methods, the feed (i.e., comprising at least one carbon source) is added in increments as the culturing process progresses. When the broth volume reaches a predefined working volume of the culture vessel, a portion of the broth is removed, generating new vessel capacity to accommodate further carbon source feeding. The repeated fed-batch systems are useful to maximize culture vessel capacity and enable the production of more total product than the standard fed-batch process.

As used herein, “fed-batch method” refers to a method by which a fed-batch culture or repeated fed-batch culture is supplied with additional nutrients. For example, in some embodiments, fed-batch methods (including repeated fed-batch methods) comprise adding supplemental media according to a determined feeding schedule within a given time period.

As used herein, “feed” refers to any addition of any substance (e.g., any desired component[s]) provided to a culture after inoculation. In some embodiments, feeding involves one addition, while in other embodiments, feeding involves two, three, four, or more additions.

As used herein, the terms “feed solution,” “feed medium,” and “feeding medium” refer to a medium containing one or more desired components that is added to the culture beginning at some time after inoculation of the production medium with the organisms (e.g., M. thermophila). In some embodiments, the feed solution comprises at least one carbon source. In some further embodiments, the carbon source comprises glucose, while in some other embodiments, the carbon source is a compound other than glucose.

As used herein, the term “feedback control system” refers to a process of monitoring a given parameter, whereby an additional agent is added or an environmental modification of the culture is performed in order to meet a desired parameter setpoint. In some embodiments, the parameter is the broth volume in the culture vessel, while in some other embodiments, the parameter is the glucose concentration in the medium. Feedback control systems find use in maintaining nutritional components needed to optimize protein production by cultures.

As used herein, “feed profile” refers to a schedule for supplementing a culture with a feed solution. In some embodiments, the feed profile is generated using a feedback control system.

As used herein, the terms “inducer” and “inducing compound” refer to any molecule or compound that positively influences the over-production of any protein (e.g., enzyme) over the corresponding basal level of production.

As used herein, the term “inducer-free” media refers to media that lack any inducer molecule or compound, while the term “inducer-containing” media refers to media that comprise one or more inducers.

“Continuous culturing” is an open system where a defined culture medium is added continuously to a bioreactor and an equal amount of conditioned medium is removed simultaneously for processing. Continuous culturing generally maintains the cultures at a constant high density where cells are primarily in log phase growth. Continuous culturing systems strive to maintain steady state growth conditions. Methods for modulating nutrients and growth factors for continuous culturing processes as well as techniques for maximizing the rate of product formation are well known in the art of industrial microbiology.

In some embodiments, the cellulase enzyme mixtures of the present invention are produced in a culturing process in which the fungal cell described herein above is grown in a submerged liquid culture. It is intended that any suitable culture medium and process will find use in the present invention. In some embodiments, submerged liquid cultures of fungal cells are conducted as a batch, fed-batch and/or continuous process. It is not intended that the present invention be limited to any particular culture medium, protocol, process, and/or equipment. In some embodiments, the culture medium is a liquid comprising a carbon source, a nitrogen source, and other nutrients, vitamins and minerals which can be added to the culture medium to improve growth and enzyme production of the host cell. In some embodiments, these other media components are added prior to, simultaneously with or after inoculation of the culture with the host cell. In some embodiments, the carbon source comprises a carbohydrate that induces the expression of the cellulase enzymes from the fungal cell. For example, in some embodiments, the carbon source comprises one or more of cellulose, cellobiose, sophorose, xylan, xylose, xylobiose and related oligo- or poly-saccharides known to induce expression of cellulases and beta-glucosidase in such fungal cells. In some embodiments, various media and carbon sources find use for growing fungi (e.g., M. thermophila) in submerged cultures. For example, standard fungal media like PDB (Sigma Aldrich), TSB (BD BioSciences), Czapek Dox (Thermo Scientific-Oxoid), Malt Extract media (Thermo Scientific-Oxoid), etc., find use. Growth of M. thermophila in submerged cultures containing various carbon sources, including but not limited to monosaccharides, disaccharides, polysaccharides (e.g., dextrins), polyols, complex carbon sources, molasses, oils, vegetable oils, palm oil, nut oils, glucose, fructose, galactose, lactose, xylose, sucrose, cellobiose, glycerol, cellulose, mannose, fructose, ribose, xylose, arabinose, rhamnose, galacturonic acid, glucuronic acid, cellobiose, maltose, lactose, raffinose, sucrose, arabinogalactan, beechwood xylan, birchwood xylan, oat spelt xylan, arabic gum, guar gum, soluble starch, apple pectin, citrus pectin, inulin, lignin, wheat bran, sugar beet pulp, citrus pulp, soybean hulls, rice bran, cotton seed pulp, alfalfa meal, casein, cellulose, starch, wheat bran, oat-spelt xylan, wheat straw, cotton, corn products, rice straw, sugarcane bagasse, paddy straw, paddy husk, grass, sugar beet pulp, sugar beets, filter paper, carboxy-methyl cellulose, etc., are known in the art (See e.g., Dubey and Johri, Proc. Indian Acad. Sci. (Plant Sci.), 97:247 [1987]; Grajek, Enz. Microb. Technol., 9:744 [1987]; Sen et al., Can. J. Microbiol., 29:1258 [1983]; and Svistova et al., Mikrobiol., 55(1):49 [1986]). Indeed it is intended that any suitable medium will find use in the present invention. Furthermore, in some embodiments, the media comprise cellulose, while in some other embodiments, the media do not comprise cellulose (i.e., measurable concentrations of cellulose). In some additional embodiments, the media comprise carbon sources such as glucose, dextrose, etc. However, it is not intended that the present invention be limited to any specific carbon and/or nitrogen source, as any suitable carbon and/or nitrogen source finds use in the present invention. It is not intended that the present invention be limited to any particular medium, as any suitable medium will find use in the desired setting.

In some embodiments utilizing batch culturing methods, the carbon source is added to the medium prior to or simultaneously with inoculation. In some other embodiments utilizing fed-batch and/or continuous operations, the carbon source is also supplied continuously or intermittently during culturing process. For example, in some embodiments, the carbon source is supplied at a carbon feed rate of between about 0.2 and about 10 g carbon/L of culture/h, or any amount therebetween. In some additional embodiments, the carbon source is supplied at a feed rate of between about 0.1 and about 10 g carbon/L of culture/hour or at any suitable rate therebetween (e.g., about 0.15 about 0.2, about 0.3, about 0.4, about 0.5, about 0.6, about 0.7, about 0.8, about 0.9, about 1, about 2, about 3, about 4, about 5, about 6, about 7, about 8, about 9, or about 10 g carbon/L of culture/h).

In some embodiments, the process for producing the enzyme mixture of the present invention is performed at a temperature from about 20° C. to about 80° C., or any temperature therebetween, for example from about 25° C. to about 65° C., or any temperature therebetween, or about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C.

In some embodiments, the methods for producing enzyme mixtures of the present invention are carried out at a pH from about 3.0 to about 8.0, or any pH therebetween, for example from about pH 3.5 to about pH 6.8, or any pH therebetween, for example, about pH 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, 6.5, about 6.6, about 6.7, about 6.8, about 6.9, about 7.0, about 7.1, about 7.2, about 7.3, about 7.4, 7.5, about 7.6, about 7.7, about 7.8, about 7.9, or about 8.0.

In some embodiments, the culture medium containing the fungal cells is used following the culturing process, while in some other embodiments, the medium containing the fungal cells and the enzyme mixture is used, while in some additional embodiments, an enzyme mixture is separated from the fungal cells (e.g., by filtration and/or centrifugation), and the enzyme mixture in the culture medium is used, and in still additional embodiments, the fungal cells, enzyme(s), and/or enzyme mixtures are separated from the culture medium and then used. Low molecular solutes such as unconsumed components of the culture medium may be removed by ultrafiltration or any other suitable method. Any suitable method for separating cells, enzyme(s), and/or enzyme mixtures find use in the present invention. Indeed, it is not intended that the present invention be limited to any particular purification/separation method. In some additional embodiments, the fungal cells, enzyme(s) and/or enzyme mixtures are concentrated (e.g., by evaporation, precipitation, sedimentation and/or filtration). In some embodiments, stabilizers are added to the compositions comprising fungal cells, enzyme(s), and/or enzyme mixtures. In some embodiments, chemicals such as glycerol, sucrose, sorbitol and the like find use to stabilize the enzyme mixtures. In some additional embodiments, other chemicals (e.g., sodium benzoate and/or potassium sorbate), are added to the enzyme mixture to prevent growth of microbial contamination. In some additional embodiments, additional components are present in the compositions provided herein. It is not intended that the present invention be limited to any particular chemical and/or other components, as various components will find use in different settings. Indeed, it is contemplated that any suitable component will find use in the compositions of the present invention.

As used herein, the term “C1” refers to Myceliophthora thermophila, including the fungal strain described by Garg (See, Garg, Mycopathol., 30: 3-4 [1966]). As used herein, “Chrysosporium lucknowense” includes the strains described in U.S. Pat. Nos. 6,015,707, 5,811,381 and 6,573,086; US Pat. Pub. Nos. 2007/0238155, US 2008/0194005, US 2009/0099079; International Pat. Pub. Nos., WO 2008/073914 and WO 98/15633, all of which are incorporated herein by reference, and include, without limitation, Chrysosporium lucknowense Garg 27K, VKM-F 3500 D (Accession No. VKM F-3500-D), C1 strain UV13-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Although initially described as Chrysosporium lucknowense, C1 may currently be considered a strain of Myceliophtora thermophila. Other C1 strains include cells deposited under accession numbers ATCC 44006, CBS (Centraalbureau voor Schimmelcultures) 122188, CBS 251.72, CBS 143.77, CBS 272.77, CBS 122190, CBS 122189, and VKM F-3500D. Exemplary C1 derivatives include modified organisms in which one or more endogenous genes or sequences have been deleted or modified and/or one or more heterologous genes or sequences have been introduced. Derivatives include, but are not limited to UV18#100f Δalp1, UV18#100f Δpyr5 Δalp1, UV18#100.f Δalp1 Δpep4 Δalp2, UV18#100.f Δpyr5 Δalp1 Δpep4 Δalp2 and UV18#100.f Δpyr4 Δpyr5 ΔaIp1 Δpep4 Δalp2, as described in WO2008073914 and WO02010107303, each of which is incorporated herein by reference. An additional M. thermophila strain has been deposited as ATCC PTA-12255.

As used herein, the terms “improved thermoactivity” and “increased thermoactivity” refer to an enzyme (e.g., a “test” enzyme of interest) displaying an increase, relative to a reference enzyme, in the amount of enzymatic activity (e.g., substrate hydrolysis) in a specified time under specified reaction conditions, for example, elevated temperature.

As used herein, the terms “improved thermostability” and “increased thermostability” refer to an enzyme (e.g., a “test” enzyme of interest) displaying an increase in “residual activity” relative to a reference enzyme. Residual activity is determined by (1) exposing the test enzyme or reference enzyme to stress conditions of elevated temperature, optionally at lowered pH, for a period of time and then determining EG1b activity; (2) exposing the test enzyme or reference enzyme to unstressed conditions for the same period of time and then determining EG1b activity; and (3) calculating residual activity as the ratio of activity obtained under stress conditions (1) over the activity obtained under unstressed conditions (2). For example, the EG1b activity of the enzyme exposed to stress conditions (“a”) is compared to that of a control in which the enzyme is not exposed to the stress conditions (“b”), and residual activity is equal to the ratio a/b. A test enzyme with increased thermostability will have greater residual activity than the reference enzyme. In some embodiments, the enzymes are exposed to stress conditions of 55° C. at pH 5.0 for 1 hr, but other cultivation conditions can be used.

As used herein, the term “culturing” refers to growing a population of microbial cells under suitable conditions in a liquid or solid medium.

As used herein, the term “saccharification” refers to the process in which substrates (e.g., cellulosic biomass) are broken down via the action of cellulases to produce fermentable sugars (e.g. monosaccharides such as but not limited to glucose).

As used herein, the term “fermentable sugars” refers to simple sugars (e.g., monosaccharides, disaccharides and short oligosaccharides), including but not limited to glucose, xylose, galactose, arabinose, mannose and sucrose. Indeed, a fermentable sugar is any sugar that a microorganism can utilize or ferment.

As used herein the term “soluble sugars” refers to water-soluble hexose monomers and oligomers of up to about six monomer units.

As used herein, the term “fermentation” is used broadly to refer to the cultivation of a microorganism or a culture of microorganisms that use simple sugars, such as fermentable sugars, as an energy source to obtain a desired product.

The terms “biomass,” and “biomass substrate,” encompass any suitable materials for use in saccharification reactions. The terms encompass, but are not limited to materials that comprise cellulose (i.e., “cellulosic biomass,” “cellulosic feedstock,” and “cellulosic substrate”). Biomass can be derived from plants, animals, or microorganisms, and may include, but is not limited to agricultural, industrial, and forestry residues, industrial and municipal wastes, and terrestrial and aquatic crops grown for energy purposes. Examples of biomass substrates include, but are not limited to, wood, wood pulp, paper pulp, corn fiber, corn grain, corn cobs, crop residues such as corn husks, corn stover, grasses, wheat, wheat straw, barley, barley straw, hay, rice, rice straw, switchgrass, waste paper, paper and pulp processing waste, woody or herbaceous plants, fruit or vegetable pulp, fruit pods, distillers grain, grasses, rice hulls, cotton, hemp, flax, sisal, sugar cane bagasse, sorghum, soy, cereal straw, switchgrass, components obtained from milling of grains, trees, branches, roots, leaves, wood chips, sawdust, shrubs and bushes, vegetables, fruits, and flowers and any suitable mixtures thereof. In some embodiments, the biomass comprises, but is not limited to cultivated crops (e.g., grasses, including C4 grasses, such as switch grass, cord grass, rye grass, miscanthus, reed canary grass, or any combination thereof), sugar processing residues, for example, but not limited to, bagasse (e.g., sugar cane bagasse, beet pulp [e.g., sugar beet], or a combination thereof), vinasse (e.g., cane-vinasse, beet-vinasse, or a combination thereof, etc.), agricultural residues (e.g., soybean stover, corn stover, corn fiber, rice straw, sugar cane straw, rice, rice hulls, barley straw, corn cobs, wheat straw, canola straw, oat straw, oat hulls, corn fiber, hemp, flax, sisal, cotton, or any combination thereof), fruit pulp, vegetable pulp, distillers' grains, forestry biomass (e.g., wood, wood pulp, paper pulp, recycled wood pulp fiber, sawdust, hardwood, such as aspen wood, softwood, or a combination thereof). Furthermore, in some embodiments, the biomass comprises cellulosic waste material and/or forestry waste materials, including but not limited to, paper and pulp processing waste, municipal paper waste, municipal solid waste, newsprint, cardboard and the like. In some embodiments, biomass comprises one species of fiber, while in some alternative embodiments, the biomass comprises a mixture of fibers that originate from different biomasses. In some embodiments, the biomass may also comprise transgenic plants that express ligninase and/or cellulase enzymes (See e.g., US 2008/0104724 A1).

A biomass substrate is said to be “pretreated” when it has been processed by some physical (i.e., mechanical), biological, and/or chemical means to release and/or separate cellulose, hemicelluloses, and/or lignin, thereby facilitating saccharification. As described further herein, in some embodiments, the biomass substrate is “pretreated,” or treated using methods known in the art, such as chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms) and combinations thereof, to increase the susceptibility of cellulose to hydrolysis. In some embodiments, the pre-treated biomass is washed and/or detoxified prior to or after hydrolysis.

The term “biomass” encompasses any living or dead biological material that contains a polysaccharide substrate, including but not limited to cellulose, starch, other forms of long-chain carbohydrate polymers, and mixtures of such sources. It may or may not be assembled entirely or primarily from glucose or xylose, and may optionally also contain various other pentose or hexose monomers. Xylose is an aldopentose containing five carbon atoms and an aldehyde group. It is the precursor to hemicellulose, and is often a main constituent of biomass. In some embodiments, the substrate is slurried prior to pretreatment. In some embodiments, the consistency of the slurry is between about 2% and about 30% and more typically between about 4% and about 15%. In some embodiments, the slurry is subjected to a water and/or acid soaking operation prior to pretreatment. In some embodiments, the slurry is dewatered using any suitable method to reduce steam and chemical usage prior to pretreatment. Examples of dewatering devices include, but are not limited to pressurized screw presses (See e.g., WO 2010/022511, incorporated herein by reference) pressurized filters and extruders.

In some embodiments, the pretreatment is carried out to hydrolyze hemicellulose, and/or a portion thereof present in the cellulosic substrate to monomeric pentose and hexose sugars (e.g., xylose, arabinose, mannose, galactose, and/or any combination thereof). In some embodiments, the pretreatment is carried out so that nearly complete hydrolysis of the hemicellulose and a small amount of conversion of cellulose to glucose occurs. In some embodiments, an acid concentration in the aqueous slurry from about 0.02% (w/w) to about 2% (w/w), or any amount therebetween, is typically used for the treatment of the cellulosic substrate. Any suitable acid finds use in these methods, including but not limited to, hydrochloric acid, nitric acid, and/or sulfuric acid. In some embodiments, the acid used during pretreatment is sulfuric acid. Steam explosion is one method of performing acid pretreatment of biomass substrates (See e.g., U.S. Pat. No. 4,461,648). Another method of pretreating the slurry involves continuous pretreatment (i.e., the cellulosic biomass is pumped though a reactor continuously). This methods are well-known to those skilled in the art (See e.g., U.S. Pat. No. 7,754,457).

In some embodiments, alkali is used in the pretreatment. In contrast to acid pretreatment, pretreatment with alkali may not hydrolyze the hemicellulose component of the biomass. Rather, the alkali reacts with acidic groups present on the hemicellulose to open up the surface of the substrate. In some embodiments, the addition of alkali alters the crystal structure of the cellulose so that it is more amenable to hydrolysis. Examples of alkali that find use in the pretreatment include, but are not limited to ammonia, ammonium hydroxide, potassium hydroxide, and sodium hydroxide. One method of alkali pretreatment is Ammonia Freeze Explosion, Ammonia Fiber Explosion or Ammonia Fiber Expansion (“AFEX” process; See e.g., U.S. Pat. Nos. 5,171,592; 5,037,663; 4,600,590; 6,106,888; 4,356,196; 5,939,544; 6,176,176; 5,037,663 and 5,171,592). During this process, the cellulosic substrate is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. In some embodiments, the flashed ammonia is then recovered using methods known in the art. In some alternative methods, dilute ammonia pretreatment is utilized. The dilute ammonia pretreatment method utilizes more dilute solutions of ammonia or ammonium hydroxide than AFEX (See e.g., WO2009/045651 and US 2007/0031953). This pretreatment process may or may not produce any monosaccharides.

An additional pretreatment process for use in the present invention includes chemical treatment of the cellulosic substrate with organic solvents, in methods such as those utilizing organic liquids in pretreatment systems (See e.g., U.S. Pat. No. 4,556,430; incorporated herein by reference). These methods have the advantage that the low boiling point liquids easily can be recovered and reused. Other pretreatments, such as the Organosolv™ process, also use organic liquids (See e.g., U.S. Pat. No. 7,465,791, which is also incorporated herein by reference). Subjecting the substrate to pressurized water may also be a suitable pretreatment method (See e.g., Weil et al. (1997) Appl. Biochem. Biotechnol., 68(1-2): 21-40 [1997], which is incorporated herein by reference). In some embodiments, the pretreated cellulosic biomass is processed after pretreatment by any of several steps, such as dilution with water, washing with water, buffering, filtration, detoxification (e.g., steam stripping, evaporation, ion exchange resin, and/or charcoal treatment; See also, WO 2008/076738 and WO 2008/13454) or centrifugation, or any combination of these processes, prior to enzymatic hydrolysis, as is familiar to those skilled in the art. The pretreatment produces a pretreated feedstock composition (e.g., a “pretreated feedstock slurry”) that contains a soluble component including the sugars resulting from hydrolysis of the hemicellulose, optionally acetic acid and other inhibitors, and solids including unhydrolyzed feedstock and lignin. In some embodiments, the soluble components of the pretreated feedstock composition are separated from the solids to produce a soluble fraction. In some embodiments, the soluble fraction, including the sugars released during pretreatment and other soluble components (e.g., inhibitors), is then sent to fermentation. However, in some embodiments in which the hemicellulose is not effectively hydrolyzed during the pretreatment one or more additional steps are included (e.g., a further hydrolysis step(s) and/or enzymatic treatment step(s) and/or further alkali and/or acid treatment) to produce fermentable sugars. In some embodiments, the separation is carried out by washing the pretreated feedstock composition with an aqueous solution to produce a wash stream and a solids stream comprising the unhydrolyzed, pretreated feedstock. Alternatively, the soluble component is separated from the solids by subjecting the pretreated feedstock composition to a solids-liquid separation, using any suitable method (e.g., centrifugation, microfiltration, plate and frame filtration, cross-flow filtration, pressure filtration, vacuum filtration, etc.). Optionally, in some embodiments, a washing step is incorporated into the solids-liquids separation. In some embodiments, the separated solids containing cellulose, then undergo enzymatic hydrolysis with cellulase enzymes in order to convert the cellulose to glucose. In some embodiments, the pretreated feedstock composition is fed into the fermentation process without separation of the solids contained therein. In some embodiments, the unhydrolyzed solids are subjected to enzymatic hydrolysis with cellulase enzymes to convert the cellulose to glucose after the fermentation process. In some embodiments, the pretreated cellulosic feedstock is subjected to enzymatic hydrolysis with cellulase enzymes.

As used herein, the term “lignocellulosic biomass” refers to any plant biomass comprising cellulose and hemicellulose, bound to lignin. In some embodiments, the biomass may optionally be pretreated to increase the susceptibility of cellulose to hydrolysis by chemical, physical and biological pretreatments (such as steam explosion, pulping, grinding, acid hydrolysis, solvent exposure, and the like, as well as combinations thereof). Various lignocellulosic feedstocks find use, including those that comprise fresh lignocellulosic feedstock, partially dried lignocellulosic feedstock, fully dried lignocellulosic feedstock, and/or any combination thereof. In some embodiments, lignocellulosic feedstocks comprise cellulose in an amount greater than about 20%, more preferably greater than about 30%, more preferably greater than about 40% (w/w). For example, in some embodiments, the lignocellulosic material comprises from about 20% to about 90% (w/w) cellulose, or any amount therebetween, although in some embodiments, the lignocellulosic material comprises less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% cellulose (w/w). Furthermore, in some embodiments, the lignocellulosic feedstock comprises lignin in an amount greater than about 10%, more typically in an amount greater than about 15% (w/w). In some embodiments, the lignocellulosic feedstock comprises small amounts of sucrose, fructose and/or starch. The lignocellulosic feedstock is generally first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. In some embodiments, at least 90% by weight of the particles produced from the size reduction have lengths less than between about 1/16 and about 4 in (the measurement may be a volume or a weight average length). In some embodiments, the equipment used to reduce the particle size reduction is a hammer mill or shredder. Subsequent to size reduction, the feedstock is typically slurried in water, as this facilitates pumping of the feedstock. In some embodiments, lignocellulosic feedstocks of particle size less than about 6 inches do not require size reduction.

As used herein, the term “lignocellulosic feedstock” refers to any type of lignocellulosic biomass that is suitable for use as feedstock in saccharification reactions.

As used herein, the term “pretreated lignocellulosic feedstock,” refers to lignocellulosic feedstocks that have been subjected to physical and/or chemical processes to make the fiber more accessible and/or receptive to the actions of cellulolytic enzymes, as described above.

As used herein, the term “recovered” refers to the harvesting, isolating, collecting, or recovering of protein from a cell and/or culture medium. In the context of saccharification, it is used in reference to the harvesting of fermentable sugars produced during the saccharification reaction from the culture medium and/or cells. In the context of culturing and/or fermentation, it is used in reference to harvesting the culture and/or fermentation product from the culture medium and/or cells. Thus, a process can be said to comprise “recovering” a product of a reaction (such as a soluble sugar recovered from saccharification) if the process includes separating the product from other components of a reaction mixture subsequent to at least some of the product being generated in the reaction.

As used herein, the term “slurry” refers to an aqueous solution in which are dispersed one or more solid components, such as a cellulosic substrate.

As used herein, “increasing” the yield of a product (such as a fermentable sugar) from a reaction occurs when a particular component of interest is present during the reaction (e.g., EG1b) causes more product to be produced, compared with a reaction conducted under the same conditions with the same substrate and other substituents, but in the absence of the component of interest (e.g., without EG1b).

As used herein, a reaction is said to be “substantially free” of a particular enzyme if the amount of that enzyme compared with other enzymes that participate in catalyzing the reaction is less than about 2%, about 1%, or about 0.1% (wt/wt).

As used herein, “fractionating” a liquid (e.g., a culture broth) means applying a separation process (e.g., salt precipitation, column chromatography, size exclusion, and filtration) or a combination of such processes to provide a solution in which a desired protein (such as an EG1b protein, a cellulase enzyme, and/or a combination thereof) comprises a greater percentage of total protein in the solution than in the initial liquid product.

As used herein, the term “enzymatic hydrolysis”, refers to a process comprising at least one cellulase and at least one glycosidase enzyme and/or a mixture glycosidases that act on polysaccharides, (e.g., cellulose), to convert all or a portion thereof to fermentable sugars. “Hydrolyzing” cellulose or other polysaccharide occurs when at least some of the glycosidic bonds between two monosaccharides present in the substrate are hydrolyzed, thereby detaching from each other the two monomers that were previously bonded.

It is intended that the enzymatic hydrolysis be carried out with any suitable type of cellulase enzymes capable of hydrolyzing the cellulose to glucose, regardless of their source, including those obtained from fungi, such as Trichoderma spp., Aspergillus spp., Hypocrea spp., Humicola spp., Neurospora spp., Orpinomyces spp., Gibberella spp., Emericella spp., Chaetomium spp., Chrysosporium spp., Fusarium spp., Penicillium spp., Magnaporthe spp., Phanerochaete spp., Trametes spp., Lentinula edodes, Gleophyllumn trabeiu, Ophiostoma piliferum, Corpinus cinereus, Geomyces pannorum, Cryptococcus laurentii, Aureobasidium pullulans, Amorphotheca resinae, Leucosporidium scotti, Cunninghamella elegans, Thermomyces lanuginosus, Myceliophthora thermophila, and Sporotrichum thermophile, as well as those obtained from bacteria of the genera Bacillus, Thermomyces, Clostridium, Streptomyces and Thermobifida. Cellulase compositions typically comprise one or more cellobiohydrolase, endoglucanase, and beta-glucosidase enzymes. In some cases, the cellulase compositions additionally contain hemicellulases, esterases, swollenins, cips, etc. Many of these enzymes are readily commercially available.

In some embodiments, the enzymatic hydrolysis is carried out at a pH and temperature that is at or near the optimum for the cellulase enzymes being used. For example, the enzymatic hydrolysis may be carried out at about 30° C. to about 75° C., or any suitable temperature therebetween, for example a temperature of about 30° C., about 35° C., about 40° C., about 45° C., about 50° C., about 55° C., about 60° C., about 65° C., about 70° C., about 75° C., or any temperature therebetween, and a pH of about 3.5 to about 7.5, or any pH therebetween (e.g., about 3.5, about 4.0, about 4.5, about 5.0, about 5.5, about 6.0, about 6.5, about 7.0, about 7.5, or any suitable pH therebetween). In some embodiments, the initial concentration of cellulose, prior to the start of enzymatic hydrolysis, is preferably about 0.1% (w/w) to about 20% (w/w), or any suitable amount therebetween (e.g., about 0.1%, about 0.5%, about 1%, about 2%, about 4%, about 6%, about 8%, about 10%, about 12%, about 14%, about 15%, about 18%, about 20%, or any suitable amount therebetween.) In some embodiments, the combined dosage of all cellulase enzymes is about 0.001 to about 100 mg protein per gram cellulose, or any suitable amount therebetween (e.g., about 0.001, about 0.01, about 0.1, about 1, about 5, about 10, about 15, about 20, about 25, about 30, about 40, about 50, about 60, about 70, about 80, about 90, about 100 mg protein per grain cellulose or any amount therebetween. The enzymatic hydrolysis is carried out for any suitable time period. In some embodiments, the enzymatic hydrolysis is carried out for a time period of about 0.5 hours to about 200 hours, or any time therebetween (e.g., about 2 hours to about 100 hours, or any suitable time therebetween). For example, in some embodiments, it is carried out for about 0.5, about 1, about 2, about 5, about 7, about 10, about 12, about 14, about 15, about 20, about 25, about 30, about 35, about 40, about 45, about 50, about 55, about 60, about 65, about 70, about 75, about 80, about 85, about 90, about 95, about 100, about 120, about 140, about 160, about 180, about 200, or any suitable time therebetween.)

In some embodiments, the enzymatic hydrolysis is batch hydrolysis, continuous hydrolysis, and/or a combination thereof. In some embodiments, the hydrolysis is agitated, unmixed, or a combination thereof. The enzymatic hydrolysis is typically carried out in a hydrolysis reactor. The cellulase enzyme composition is added to the pretreated lignocellulosic substrate prior to, during, or after the addition of the substrate to the hydrolysis reactor. Indeed it is not intended that reaction conditions be limited to those provided herein, as modifications are well-within the knowledge of those skilled in the art. In some embodiments, following cellulose hydrolysis, any insoluble solids present in the resulting lignocellulosic hydrolysate, including but not limited to lignin, are removed using conventional solid-liquid separation techniques prior to any further processing. In some embodiments, these solids are burned to provide energy for the entire process.

As used herein, the term “by-product” refers to an organic molecule that is an undesired product of a particular process (e.g., saccharification).

### DETAILED DESCRIPTION OF THE INVENTION

The present invention provides compositions and methods for the production of enzymes.

Fungi, bacteria, and other organisms produce a variety of cellulases and other enzymes that act in concert to catalyze decrystallization and hydrolysis of cellulose to yield fermentable sugars. One such fungus is M. thermophila, which is described herein. Indeed, in some embodiments of the present invention, the filamentous fungal host cells are Myceliophthora sp., and/or teleomorphs, or anamorphs, and synonyms, basionyms, or taxonomic equivalents thereof. Many strains that find use in the present invention are readily available to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. For example, knockout of Alp1 function results in a cell that is protease deficient. Knockout of pyr5 function results in a cell with a pyrimidine deficient phenotype. In some embodiments, the host cells are modified to delete endogenous cellulase protein-encoding sequences or otherwise eliminate expression of one or more endogenous cellulases. In some embodiments, expression of one or more endogenous cellulases is inhibited to increase production of cellulases of interest. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of cellulase(s) within the host cell and/or in the culture medium. For example, knockout of Alp1 function results in a cell that is protease deficient, and knockout of pyr5 function results in a cell with a pyrimidine deficient phenotype. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression.

In some embodiments, host cells (e.g., Myceliophthora thermophila) used for cellulase production have been genetically modified to reduce the amount of endogenous cellobiose dehydrogenase (EC 1.1.3.4) and/or other enzymes activity that is secreted by the cell. A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., Nucl. Acids Res., 28:22 e97 [2000]; Cho et al., Mol. Plant Micr. Interact., 19:1:7-15 [2006]; Maruyama and Kitamoto, Biotechnol. Lett., 30:1811-1817 [2008]; Takahashi et al., Mol. Gen. Genom., 272: 344-352 [2004]; and You et al., Arch. Microbiol., 191:615-622 [2009], all of which are incorporated by reference herein). Random mutagenesis, followed by screening for desired mutations also finds use (See e.g., Combier et al., FEMS Microbiol. Lett., 220:141-8 [2003]; and Firon et al., Eukary. Cell 2:247-55 [2003], both of which are incorporated by reference). In some embodiments, the host cell is modified to reduce production of endogenous cellobiose dehydrogenases. In some embodiments, the cell is modified to reduce production of cellobiose dehydrogenase (e.g., CDH1 or CDH2). In some embodiments, the host cell has less than 75%, sometimes less than 50%, sometimes less than 30%, sometimes less than 25%, sometimes less than 20%, sometimes less than 15%, sometimes less than 10%, sometimes less than 5%, and sometimes less than 1% of the cellobiose dehydrogenase (e.g., CDH1 and/or CDH2) activity of the corresponding cell in which the gene is not disrupted. Exemplary Myceliophthora thermophila cellobiose dehydrogenases include, but are not limited to CDH1 and CDH2. The genomic sequence for the Cdh1 encoding CDH1 has accession number AF074951.1. In one approach, gene disruption is achieved using genomic flanking markers (See e.g., Rothstein, Meth. Enzymol., 101:202-11 [1983]). In some embodiments, site-directed mutagenesis is used to target a particular domain of a protein, in some cases, to reduce enzymatic activity (e.g., glucose-methanol-choline oxido-reductase N and C domains of a cellobiose dehydrogenase or heme binding domain of a cellobiose dehydrogenase; See e.g., Rotsaert et al., Arch. Biochem. Biophys., 390:206-14 [2001], which is incorporated by reference herein in its entirety).

Introduction of a vector or DNA construct into a host cell can be accomplished using any suitable method known in the art, including but not limited to calcium phosphate transfection, DEAE-Dextran mediated transfection, PEG-mediated transformation, electroporation, or other common techniques known in the art.

The present invention provides methods of producing at least one polypeptides or biologically active fragments thereof. In some embodiments, the method comprises: providing a host cell transformed with a polynucleotide encoding an amino acid sequence comprising at least one polypeptide; culturing the transformed host cell in a culture medium under conditions in which the host cell expresses the encoded polypeptide(s); and optionally recovering or isolating the expressed polypeptide(s), and/or recovering or isolating the culture medium containing the expressed polypeptide(s). In some embodiments, the methods further provide optionally lysing the transformed host cells after expressing the encoded polypeptide(s) and optionally recovering and/or isolating the expressed polypeptide(s) from the cell lysate. Typically, recovery or isolation of the cellulase polypeptide(s) is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including, but not limited to freeze-thaw cycling, sonication, mechanical disruption, and/or use of cell lysing agents, as well as many other methods, which are well known to those skilled in the art.

In some embodiments, the resulting polypeptide is recovered/isolated and optionally purified by any of a number of methods known in the art. For example, in some embodiments, the polypeptide is isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. For example, the methods for purifying BGL1 known in the art, find use in the present invention (See e.g., Parry et al., Biochem. J., 353:117 [2001]; and Hong et al., Appl. Microbiol. Biotechnol., 73:1331 [2007], both incorporated herein by reference). Indeed, any suitable purification methods known in the art find use in the present invention.

In some embodiments, immunological methods are used to purify the cellulase(s). In one approach, antibody raised against a cellulase polypeptide, using conventional methods is immobilized on beads, mixed with cell culture media under conditions in which the cellulase is bound, and precipitated. In a related approach, immunochromatography finds use.

In some embodiments, the cellulase is expressed as a fusion protein including a non-enzyme portion. In some embodiments, the cellulase sequence is fused to a purification facilitating domain. As used herein, the term “purification facilitating domain” refers to a domain that mediates purification of the polypeptide to which it is fused. Suitable purification domains include, but are not limited to metal chelating peptides, histidine-tryptophan modules that allow purification on immobilized metals, a sequence which binds glutathione (e.g., GST), a hemagglutinin (HA) tag (corresponding to an epitope derived from the influenza hemagglutinin protein; See e.g., Wilson et al., Cell 37:767 [1984]), maltose binding protein sequences, the FLAG epitope utilized in the FLAGS extension/affinity purification system (e.g., the system available from Immunex Corp, Seattle, Wash.), and the like. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography; See e.g., Porath et al., Prot. Exp. Purif., 3:263-281 [1992]) while the enterokinase cleavage site provides a means for separating the cellulase polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

In some embodiments, an “end-product of fermentation” is any product produced by a process including a fermentation step using a fermenting organism. Examples of end-products of a fermentation include, but are not limited to, alcohols (e.g., fuel alcohols such as ethanol and butanol), organic acids (e.g., citric acid, acetic acid, lactic acid, gluconic acid, and succinic acid), glycerol, ketones, diols, amino acids (e.g., glutamic acid), antibiotics (e.g., penicillin and tetracycline), vitamins (e.g., beta-carotene and B12), hormones, and fuel molecules other than alcohols (e.g., hydrocarbons), and proteins.

In some embodiments, the fermentable sugars produced by the methods of the present invention are used to produce at least one alcohol (e.g., ethanol, butanol, etc.). It is not intended that the present invention be limited to the specific methods provided herein. Two methods commonly employed are separate saccharification and fermentation (SHF) methods (See e.g., Wilke et al., Biotechnol. Bioengin., 6:155-75 [1976]) and simultaneous saccharification and fermentation (SSF) methods (See e.g., U.S. Pat. Nos. 3,990,944 and 3,990,945, incorporated herein by reference). In some embodiments, the SHF saccharification method comprises the steps of contacting a cellulase with a cellulose containing substrate to enzymatically break down cellulose into fermentable sugars (e.g., monosaccharides such as glucose), contacting the fermentable sugars with an alcohol-producing microorganism to produce alcohol (e.g., ethanol or butanol) and recovering the alcohol. In some embodiments, the method of consolidated bioprocessing (CBP) finds use, in which the cellulase production from the host is simultaneous with saccharification and fermentation either from one host or from a mixed cultivation. In addition, SSF methods find use in the present invention. In some embodiments, SSF methods provide a higher efficiency of alcohol production than that provided by SHF methods (See e.g., Drissen et al., Biocat. Biotrans., 27:27-35 [2009]).

In some embodiments, for cellulosic substances to be effectively used as substrates for the saccharification reaction in the presence of a cellulase of the present invention, it is desirable to pretreat the substrate. Means of pretreating a cellulosic substrate are well-known in the art, including but not limited to chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms), and the present invention is not limited by such methods.

In some embodiments, any suitable alcohol producing microorganism known in the art (e.g., Saccharomyces cerevisiae), finds use in the present invention for the fermentation of fermentable sugars to alcohols and other end-products. The fermentable sugars produced from the use of the methods and compositions provided by the present invention find use in the production of other end-products besides alcohols, including, but not limited to biofuels and/or biofuels compounds, acetone, amino acids (e.g., glycine, lysine, etc.), organic acids (e.g., lactic acids, etc.), glycerol, ascorbic acid, diols (e.g., 1,3-propanediol, butanediol, etc.), vitamins, hormones, antibiotics, other chemicals, and animal feeds. In addition, the cellulase(s) provided herein further find use in the pulp and paper industry. Indeed, it is not intended that the present invention be limited to any particular end-products.

In some embodiments, the present invention provides enzyme mixtures, as provided herein. The enzyme mixture may be cell-free, or in alternative embodiments, may not be separated from host cells that secrete an enzyme mixture component. A cell-free enzyme mixture typically comprises enzymes that have been separated from cells. Cell-free enzyme mixtures can be prepared by any of a variety of methodologies that are known in the art, such as filtration or centrifugation methodologies. In some embodiments, the enzyme mixtures are partially cell-free, substantially cell-free, or entirely cell-free.

In some embodiments, the cellulase(s) and any additional enzymes present in the enzyme mixture are secreted from a single genetically modified fungal cell or by different microbes in combined or separate fermentations. Similarly, in additional embodiments, the cellulase(s) and any additional enzymes present in the enzyme mixture are expressed individually or in sub-groups from different strains of different organisms and the enzymes are combined in vitro to make the enzyme mixture. It is also contemplated that the cellulase(s) and any additional enzymes in the enzyme mixture will be expressed individually or in sub-groups from different strains of a single organism, and the enzymes combined to make the enzyme mixture. In some embodiments, all of the enzymes are expressed from a single host organism, such as a genetically modified fungal cell.

In some embodiments, the enzyme mixture comprises at least one cellulase, selected from cellobiohydrolase (CBH), endoglucanase (EG), glycoside hydrolase 61 (GH61) and/or beta-glucosidase (BGL) cellulase. In some embodiments, the cellobiohydrolase is T. reesei cellobiohydrolase II. In some embodiments, the endoglucanase comprises a catalytic domain derived from the catalytic domain of a Streptomyces avermitilis endoglucanase. In some embodiments, at least one cellulase is Acidothermus cellulolyticus, Thermobifida fusca, Humicola grisea, and/or a Chrysosporium sp. cellulase. Cellulase enzymes of the cellulase mixture work together in decrystallizing and hydrolyzing the cellulose from a biomass substrate to yield fermentable sugars, such as but not limited to glucose (See e.g., Brigham et al. in Wyman ([ed.], Handbook on Bioethanol, Taylor and Francis, Washington D.C. [1995], pp 119-141, incorporated herein by reference).

Some cellulase mixtures for efficient enzymatic hydrolysis of cellulose are known (See e.g., Viikari et al., Adv. Biochem. Eng. Biotechnol., 108:121-45 [2007]; and US Pat. Appln. Publns. 2009/0061484; US 2008/0057541; and US 2009/0209009, all of which are incorporated herein by reference). In some embodiments, mixtures of purified naturally occurring or recombinant enzymes are combined with cellulosic feedstock or a product of cellulose hydrolysis. In some embodiments, one or more cell populations, each producing one or more naturally occurring or recombinant cellulases, are combined with cellulosic feedstock or a product of cellulose hydrolysis.

In some embodiments, the cellulase polypeptide of the present invention is present in mixtures comprising enzymes other than cellulases that degrade cellulose, hemicellulose, pectin, and/or lignocellulose.

In some additional embodiments, the present invention provides at least one enzyme that participates in lignin degradation in an enzyme mixture. Enzymatic lignin depolymerization can be accomplished by lignin peroxidases, manganese peroxidases, laccases and cellobiose dehydrogenases (CDH), often working in synergy. These extracellular enzymes are often referred to as “lignin-modifying enzymes” or “LMEs.” Three of these enzymes comprise two glycosylated heme-containing peroxidases: lignin peroxidase (LIP); Mn-dependent peroxidase (MNP); and, a copper-containing phenoloxidase laccase (LCC).

In some additional embodiments, the present invention provides at least one cellulase and at least one protease, amylase, glucoamylase, and/or a lipase that participates in cellulose degradation.

As used herein, the term “protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the invention. Some specific types of proteases include, cysteine proteases including pepsin, papain and serine proteases including chymotrypsins, carboxypeptidases and metalloendopeptidases.

As used herein, the term “lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

In some additional embodiments, the present invention provides compositions comprising at least one expansin or expansin-like protein, such as a swollenin (See e.g., Salheimo et al., Eur. J. Biochem., 269:4202-4211 [2002]) or a swollenin-like protein. Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. In some embodiments, an expansin-like protein or swollenin-like protein comprises one or both of such domains and/or disrupts the structure of cell walls (such as disrupting cellulose structure), optionally without producing detectable amounts of reducing sugars.

In some additional embodiments, the present invention provides compositions comprising at least one polypeptide product of a cellulose integrating protein, scaffoldin or a scaffoldin-like protein, for example CipA or CipC from Clostridium thermocellum or Clostridium cellulolyticum respectively. Scaffoldins and cellulose integrating proteins are multi-functional integrating subunits which may organize cellulolytic subunits into a multi-enzyme complex. This is accomplished by the interaction of two complementary classes of domain (i.e. a cohesion domain on scaffoldin and a dockerin domain on each enzymatic unit). The scaffoldin subunit also bears a cellulose-binding module that mediates attachment of the cellulosome to its substrate. A scaffoldin or cellulose integrating protein for the purposes of this invention may comprise one or both of such domains.

In some additional embodiments, the present invention provides compositions comprising at least one cellulose induced protein or modulating protein, for example as encoded by cip1 or cip2 gene or similar genes from Trichoderma reesei (See e.g., Foreman et al., J. Biol. Chem., 278:31988-31997 [2003]).

In some additional embodiments, the present invention provides compositions comprising at least one member of each of the classes of the polypeptides described above, several members of one polypeptide class, or any combination of these polypeptide classes to provide enzyme mixtures suitable for various uses.

In some embodiments, the enzyme mixture comprises various cellulases, including, but not limited to cellobiohydrolase, endoglucanase, β-glucosidase, and glycoside hydrolase 61 protein (GH61) cellulases. These enzymes may be wild-type or recombinant enzymes. In some embodiments, the cellobiohydrolase is a type 1 cellobiohydrolase (e.g., a T. reesei cellobiohydrolase I). In some embodiments, the endoglucanase comprises a catalytic domain derived from the catalytic domain of a Streptomyces avermitilis endoglucanase (See e.g., US Pat. Appln. Pub. No. 2010/0267089, incorporated herein by reference). In some embodiments, the at least one cellulase is derived from Acidothermus cellulolyticus, Thermobifida fusca, Humicola grisea, Myceliophthora thermophila, Chaetomium thermophilum, Acremonium sp., Thielavia sp, Trichoderma reesei, Aspergillus sp., or a Chrysosporium sp. Cellulase enzymes of the cellulase mixture work together resulting in decrystailization and hydrolysis of the cellulose from a biomass substrate to yield fermentable sugars, such as but not limited to glucose.

Some cellulase mixtures for efficient enzymatic hydrolysis of cellulose are known (See e.g., Viikari et al., Adv. Biochem. Eng. Biotechnol., 108:121-45 [2007]; and US Pat. Appln. Publn. Nos. US 2009/0061484, US 2008/0057541, and US 2009/0209009, each of which is incorporated herein by reference in their entireties). In some embodiments, mixtures of purified naturally occurring or recombinant enzymes are combined with cellulosic feedstock or a product of cellulose hydrolysis. Alternatively or in addition, one or more cell populations, each producing one or more naturally occurring or recombinant cellulases, are combined with cellulosic feedstock or a product of cellulose hydrolysis.

In some embodiments, the enzyme mixture comprises commercially available purified cellulases. Commercial cellulases are known and available (e.g., C2730 cellulase from Trichoderma reesei ATCC No. 25921 available from Sigma-Aldrich, Inc.; and C9870 ACCELLERASE® 1500, available from Genencor).

In some embodiments, the enzyme component comprises more than one CBH2b, CBH1a, EG, Bgl, and/or GH61 enzyme (e.g., 2, 3 or 4 different variants), in any suitable combination. In some embodiments, an enzyme mixture composition of the invention further comprises at least one additional protein and/or enzyme. In some embodiments, enzyme mixture compositions of the present invention further comprise at least one additional enzyme other than Bgl, CBH1a, GH61, and/or CBH2b. In some embodiments, the enzyme mixture compositions of the invention further comprise at least one additional cellulase, other than the EG1b, EG2, Bgl, CBH1a, GH61, and/or CBH2b variant recited herein. In some embodiments, the EG1b polypeptide of the invention is also present in mixtures with non-cellulase enzymes that degrade cellulose, hemicellulose, pectin, and/or lignocellulose.

In some embodiments, the enzymes and enzyme mixtures of the present invention is used in combination with other optional ingredients such as at least one buffer, surfactant, and/or scouring agent. In some embodiments, at least one buffer is used to maintain a desired pH within the solution in which the EG1b is employed. The exact concentration of buffer employed depends on several factors which the skilled artisan can determine. Suitable buffers are well known in the art. In some embodiments, at least one surfactant is used in the present invention. Suitable surfactants include any surfactant compatible with the cellulase(s) and optionally, with any other enzymes being used in the mixture. Exemplary surfactants include anionic, non-ionic, and ampholytic surfactants. Suitable anionic surfactants include, but are not limited to, linear or branched alkylbenzenesulfonates; alkyl or alkenyl ether sulfates having linear or branched alkyl groups or alkenyl groups; alkyl or alkenyl sulfates; olefinsulfonates; alkanesulfonates, and the like. Suitable counter ions for anionic surfactants include, for example, alkali metal ions, such as sodium and potassium; alkaline earth metal ions, such as calcium and magnesium; ammonium ion; and alkanolamines having from 1 to 3 alkanol groups of carbon number 2 or 3. Ampholytic surfactants suitable for use in the practice of the present invention include, for example, quaternary ammonium salt sulfonates, betaine-type ampholytic surfactants, and the like. Suitable nonionic surfactants generally include polyoxalkylene ethers, as well as higher fatty acid alkanolamides or alkylene oxide adduct thereof, fatty acid glycerine monoesters, and the like. Mixtures of surfactants also find use in the present invention, as is known in the art.

The foregoing and other aspects of the invention may be better understood in connection with the following non-limiting examples.

### EXPERIMENTAL

The present invention is described in further detail in the following Examples, which are not in any way intended to limit the scope of the invention as claimed.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); DNA (deoxyribonucleic acid); RNA (ribonucleic acid); HPLC (high pressure liquid chromatography); MES (2-N-morpholino ethanesulfonic acid); FIOPC (fold improvements over positive control); YPD (10 g/L yeast extract, 20 g/L peptone, and 20 g/L dextrose); ARS (ARS Culture Collection or NRRL Culture Collection, Peoria, Ill.); ATCC (American Type Culture Collection, Manassas, Va.); ADM (Archer Daniels Midland, Decatur, Ill.); Axygen (Axygen, Inc., Union City, Calif.); Dual Biosysterns (Dual Biosystems AG, Schlieven, Switzerland); Megazyme (Megazyme International Ireland, Ltd., Wicklow, Ireland); Sigma-Aldrich (Sigma-Aldrich, St. Louis, Mo.); International Fiber (International Fiber, Corp., N. Tonawanda, N.Y.), Bussetti (Bussetti & Co., GmbH, Vienna, AT); BASF (BASF Aktiengesellschaft Corp., Ludwigshafen, Del.); Dasgip (Dasgip Biotools, LLC, Shrewsbury, Mass.); Difco (Difco Laboratories, BD Diagnostic Systems, Detroit, Mich.); PCRdiagnostics (PCRdiagnostics, by E. coli SRO, Slovak Republic); Agilent (Agilent Technologies, Inc., Santa Clara, Calif.); Molecular Devices (Molecular Devices, Sunnyvale, Calif.); Symbio (Symbio, Inc., Menlo Park, Calif.); Newport (Newport Scientific, Australia); and Bio-Rad (Bio-Rad Laboratories, Hercules, Calif.).

In the following Examples, variants of fungal strain C1 were utilized. These variants all have deletion of the cdh genes and are described in U.S. Pat. No. 8,309,328, which is hereby incorporated by reference in its entirety. In some experiments, the strains were further modified to overexpress the C1 beta-glucosidase and/or GH61.

### Example 1

### Inoculum Generation for Stirred-Tank Culturing

Shake flasks containing 300 ml media were inoculated with 2 ml frozen M. thermophila mycelial stocks. The media composition is provided in Table 1-1. However, it is noted that the components in the medium can be varied as described in Table 2-1 without abolishing protein production. The cultures were grown with shaking (200 rpm), at 35° C., until pH of the cultures started to increase. This usually required 2-3 days, depending on the vitality of the frozen inoculum. The % PCV (packed cell volume) of each of the cultures was >10% at the termination of the inoculum shake flask cultures.

TABLE 1-1
Inoculum Medium Components

| | Per 1 L |
|---|---|
| Compound | Medium |
| K₂HPO₄ anhydrous (g) | 0.5 |
| FeSO₄ * 7H₂O (7 g/l stock solution) [ml] | 1 |
| MgSO₄ * 7H₂O (g) | 0.3 |
| Corn steep solids(CSS) (g) | 12.5 |
| glucose monohydrate (g) | 20 |
| CaCO₃ (g) | 5 |
| antifoam before sterilization [ml] | 3 |

TABLE 1-2
Suitable Ranges of Inoculum Medium Components

| | Per 1 L |
|---|---|
| Compound | Medium |
| K₂HPO₄ anhydrous (g) | 0.1-2 |
| FeSO₄ * 7H₂O (7 g/l stock solution) [ml] | 0.1-3 |
| MgSO₄ * 7H₂O (g) | 0.1-0.6 |
| CSS (g) | 0-70 |
| glucose monohydrate (g) | 0-100 |
| CaCO₃ (g) | 0-10 |
| (NH₄)₂SO₄ (g) | 0-2 |
| antifoam before sterilization (ml) | 1-5 |

### Example 2

### Stirred Tank Culturing in Media Containing Low Cellulose Concentrations

The entire contents of one shake flask produced as described in Example 1 were used to inoculate stirred tank fermentors with a working volume of 5 L. The media composition is provided in Table 2-1. However, it is noted that each component in the medium can be varied as described in Table 2-2, without abolishing protein production. In these experiments, AlphaCel BH 200A (International Fiber) was used, although any suitable cellulose finds use in the present invention. Table 2-3 provides the components of the trace element solution. Culturing was carried out under pH-stat conditions, either at pH 5, or at pH 6.7. The pH levels of the cultures were controlled using a 25% NH₄OH solution. The pH can be varied between pH15.0 and pH7 during the process without significantly effecting protein production. Typically, a Sartorius Biostat® Bplus was used. Culturing was carried out at 38° C., 20% pO₂, 0.5-1 vvm (volume/volume/minute of aeration: calculated for starting volume) for a total duration of 120 h. Feeding started when the pH of the cultures started to increase at an average rate of 3 g glucose/kg/h, calculated for the initial weight. The feed composition is described in Table 2-4 and the 100× vitamin and trace element solution is described in Table 2-5. The feed was turned off when the pO₂ was less than 10% for at least 10 minutes; the feed was turned on again when the pO₂ reached the control level of 20%. The composition of the feed solution is provided in Table 2-6. However, it is noted that the feed solution components can be varied as described in Table 2-7 without abolishing protein production. The productivity of the culture at the end of incubation is provided in Table 2-8. The protein concentration was determined using BCA analysis with incubation at 37° C. for 60 min, as known in the art (e.g., Sigma-Aldrich protocols).

TABLE 2-1
Media Composition for Low Cellulose Cultures

| | Per 1 L |
|---|---|
| Component | Medium |
| (NH₄)₂SO₄ | 8.0 |
| KH₂PO₄ (g) | 1.52 |
| KCl (g) | 0.52 |
| MgSO₄, 7H₂O (g) | 0.49 |
| CaCl₂, 2H₂O (g) | 0.4 |
| CaCO₃ (g) | 5 |
| CSS (g) | 70 |
| Cellulose (AlphaCel ™ BH 200A; International Fiber) (g) | 37.25 |
| Glucose•H₂O (g) | 26.4 |
| Antifoam (Glanapon; Bussetti) before sterilization (ml) | 4 |
| Biotin (60 mg/L) stock (ml) | 0.1 |
| 1000X Minimal trace element solution (ml) | 1 |

TABLE 2-2
Composition Ranges for Media Components

| | Per 1 L |
|---|---|
| Component | Medium |
| (NH₄)₂SO₄ | 0.1-15 |
| KH₂PO₄ (g) | 0.1-2 |
| KCl (g) | 0.1-1 |
| MgSO₄, 7H₂O (g) | 0.1-1 |
| CaCl₂, 2H₂O (g) | 0.1-1 |
| CaCO₃ (g) | 0-10 |
| CSS (g) | 0-100 |
| Cellulose (AlphaCel ™ BH 200A; International Fiber) (g) | 0-100 |
| Glucose•H₂O (g) | 0-40 |
| Antifoam (Glanapon; Bussetti) before sterilization (ml) | 0.1-10 |
| Biotin (60 mg/L) stock (ml) | 0-1 |
| 1000X Minimal trace element solution (ml) | 0-5 |

TABLE 2-3
1000X Minimal Trace Element Solution Composition

| Component | Per 1 L |
|---|---|
| EDTA (added first, set pH = 8 with NaOH) (g) | 50 |
| ZnSO₄, 7H₂O (g) | 22 |
| H₃BO₃ (g) | 11 |
| MnSO₄, 7H₂O (g) | 4.3 |
| FeSO4, 7H₂O (g) | 5 |
| CoCl₂, 6H₂O (g) | 2.7 |
| CuSO₄, 5H₂O (g) | 1.6 |
| Na₂MoO₄, 2H₂O (g) | 1.5 |

TABLE 2-4
Feed Solution Composition

| Component | Per 1 L |
|---|---|
| KH₂PO₄ (g) | 0.65 |
| MgSO₄, 7H₂O (g) | 0.65 |
| (NH₄)₂SO₄ (g) | 30 |
| Dextrose, H₂O | 616 |
| Biotin (Lutavit ®; BASF: 2% biotin content) | 0.125 |
| 100X Vitamin and trace element solution (ml) | 10 |

TABLE 2-5
100 x Vitamin and Trace Element Solution

| Component | Per 1 L |
|---|---|
| EDTA (before others, set pH = 8, use NaOH) (g) | 6.5 |
| ZnSO₄*7H₂O (g) | 2.85 |
| H₃BO₃ (g) | 1.5 |
| MnSO₄*H₂O (g) | 0.25 |
| FeSO₄*7H₂O (g) | 2.5 |
| CoCl₂*6H₂O (g) | 0.22 |
| CuSO₄*5H₂O (g) | 0.25 |
| Na₂MoO₄*2H₂O (g) | 0.2 |
| NiCl₂*6H₂O (g) | 0.09 |
| Thiamine (g) | 1.0 |
| Calpan (g) | 2.5 |
| Nicotinic acid (g) | 3 |
| Sodium citrate tribasic (g) | 7.5 |

TABLE 2-6
Range of Component Concentrations in Feed Solutions

| Component | Per 1 L |
|---|---|
| KH₂PO₄ (g) | 0.1-1 |
| MgSO₄, 7H₂O (g) | 0.1-1 |
| (NH₄)₂SO₄ | 0-50 |
| Dextrose, H₂O | 200-650 |
| Biotin (Lutavit ®; BASF: 2% biotin content) | 0-1 |
| 100X Minimal trace element solution (ml) | 0-20 |

### Example 3

### Stirred Tank Culturing in Media Containing No Cellulose

The entire contents of one shake flask (produced as described in Example 1) was used to inoculate stirred tank fermentors with a working volume of 5 L. The media composition is provided in Table 2-1, except that the media did not contain any cellulose or glucose. The trace element solution used in the media is provided in Table 2-3. Culturing was carried out under pH-stat conditions, either at pH 5, or at pH 6.7. The pH levels of the cultures were controlled using a 25% NH₄OH solution. Typically, a Sartorius Biostat® Bplus was used. Culturing was carried out at 38° C., 20% pO₂, 0.5-1 vvm (volume/volume/minute of aeration; calculated for starting volume) for a total duration of 120 h. Feeding started as soon as the culture was started, at an average rate of 3 g glucose/kg/h, calculated for the initial weight. The feed was turned off when pO₂ was less than 10% for at least 10 minutes; the feed was turned on again when the pO₂ reached the control level of 20%. The composition of the feed solution is provided in Table 2-4, except that the (NH₄)₂SO₄ concentration was cut in half. The productivity of the culture at the end of the incubation is provided in Table 3-1. The protein concentration was determined using BCA analysis using incubation at 37° C. for 60 min., using methods known in the art (e.g., Sigma-Aldrich protocols).

In some additional experiments using the same media that did not contain cellulose or glucose, successful cultures were achieved in which the glucose feeding was varied between 3-5 g/kg/h. Protein productivity after 120 h of culturing for the cultures fed at 3.5 and 5 g/kg/h glucose were 85 and 88 g/L, respectively. The glucose concentration, when tested at any given point of these cultures, did not exceed 1.85 g/L.

TABLE 3.1
Protein Production

| Culture Condition | Protein Production [g/L] |
|---|---|
| Low cellulose, pH = 5 | ~76 |
| Low cellulose, pH = 6.7 | ~69 |
| No cellulose, pH = 5 | ~74 |
| No cellulose, pH = 6.7 | ~68 |

### Example 4

### Stirred Tank Cultures in Media Containing No Cellulose in the Presence of Carbon Sources Other than Glucose

The entire contents of one shake flask (produced as described in Example 1) was used to inoculate stirred tank fermentor vessels with a working volume of 5 L. The media used were the same as shown content of the media used is presented in Table 2-1, except that the media did not contain any cellulose or glucose and the CSS concentration was cut in half. The same trace element solution as shown in Table 2-3 was used in the media Culturing was carried out under pH-stat conditions at pH 5.0. The pH levels of the cultures were controlled using a 25% NH₄OH solution. Typically, a Sartorius Biostat® Bplus was used. Culturing was carried out at 38° C., 20% pO₂, 0.5-1 vvm (volume/volume/minute of aeration; calculated for starting volume) for a total duration of 120 h. The feeding started as soon as the culture was started at an average rate of 3-4 g sucrose/kg/h, calculated for the initial weight. The feed was turned off when pO₂ was less than 10% for at least 10 minutes; the feed was turned on again when the pO₂ reached the control level of 20%. The composition of the feed solution is provided in Table 2-4 except that sucrose was used instead of the glucose and the (NH₄)₂SO₄ concentration was cut in half. The productivity of the culture at the end of incubation was determined to be ˜64-77 g/L, using a standard BCA analysis method (Sigma Aldrich) except that the incubation was at 37° C. for 60 min.

While particular embodiments of the present invention have been illustrated and described, it will be apparent to those skilled in the art that various other changes and modifications can be made without departing from the spirit and scope of the present invention. Therefore, it is intended that the present invention encompass all such changes and modifications with the scope of the present invention.

The present invention has been described broadly and generically herein. Each of the narrower species and subgeneric groupings falling within the generic disclosure also form part(s) of the invention. The invention described herein suitably may be practiced in the absence of any element or elements, limitation or limitations which is/are not specifically disclosed herein. The terms and expressions which have been employed are used as terms of description and not of limitation. There is no intention that in the use of such terms and expressions, of excluding any equivalents of the features described and/or shown or portions thereof, but it is recognized that various modifications are possible within the scope of the claimed invention. Thus, it should be understood that although the present invention has been specifically disclosed by some embodiments and optional features, modification and variation of the concepts herein disclosed may be utilized by those skilled in the art, and that such modifications and variations are considered to be within the scope of the present invention and claims.

1. A method for producing at least one enzyme, comprising: providing a fungal cell, wherein said fungal cell is of the genus Myceliophthora or a taxonomically equivalent genus, wherein said cell is capable of producing said enzyme; culturing said fungal cell under conditions that said enzyme is produced by said fungal cell, wherein said conditions comprise culturing said fungal cell in a medium comprising a cellulose content less than about 50 g/L cellulose; and harvesting said enzyme.

2. (canceled)

3. The method of claim 1, wherein said culture medium comprises no cellulose.

4. The method of claim 1, wherein said culturing is conducted at a pH of about 4 to about 10.

5. The method of claim 1, wherein said culturing comprises batch, fed-batch, continuous, and/or repeated fed-batch culturing methods.

6. The method of claim 1, wherein said culturing further comprises adding a feed solution to said culture medium.

7. The method of claim 6, wherein said feed solution comprises at least one carbon source, and/or at least one nitrogen source, and/or at least one adjunct composition.

8. The method of claim 7, wherein said feed solution comprises at least one non-inducing carbon source.

9-12. (canceled)

13. The method of claim 7, wherein said adjunct composition comprises at least one reducing agent, gallic acid, surfactant, divalent metal cation, vitamin, and/or polyethylene glycol.

14. The method of claim 6, wherein said feed solution lacks an inducer compound.

15. The method of claim 1, wherein said fungal cell produces a total protein level of at least about 2.5 g/L.

16. (canceled)

17. The method of claim 1, wherein said enzyme is selected from cellobiohydrolase, endoglucanase, beta-glucosidase, glycosyl hydrolase, xylanase, glucanase, pectinase, amylase, glucoamylase, lipase, protease, esterase, glucose isomerase, glucose oxidase, and phytase.

18. (canceled)

19. A method for producing an enzyme mixture, comprising: providing a fungal cell, wherein said fungal cell is of the genus Myceliophthora or a taxonomically equivalent genus, wherein said fungal cell is capable of producing said enzyme mixture; culturing said fungal cell under conditions that said enzyme mixture is produced by said fungal cell, wherein said conditions comprise a culturing said fungal cell in a medium comprising less than about 50 g/L cellulose; and harvesting said enzyme mixture.

20. (canceled)

21. The method of claim 19, wherein said culture medium comprises no cellulose.

22. The method of claim 19, wherein said culturing is conducted at a pH of about 4 to about 10.

23. The method of claim 19, wherein said culturing comprises fed-batch, continuous, and/or repeated fed-batch culturing methods.

24. The method of claim 19, wherein said culturing further comprises adding a feed solution to said culture medium.

25. The method of claim 24, wherein said feed solution comprises at least one carbon source, and/or at least one nitrogen source, and/or at least one adjunct composition.

26. The method of claim 24, wherein said feed solution comprises at least one non-inducing carbon source.

27. The method of claim 24, wherein said feed solution does not comprise glucose.

28. The method of claim 24, wherein said feed solution comprises at least one compound selected from monosaccharides, disaccharides, polysaccharides, alcohols, molasses, polyols, glycerol, and sucrose.

29-30. (canceled)

31. The method of claim 25, wherein said adjunct composition comprises at least one reducing agent, gallic acid, surfactant, divalent metal cation, vitamin, and/or polyethylene glycol.

32. The method of claim 24, wherein said feed solution lacks an inducer compound.

33. The method of claim 19, wherein said fungal cell produces a total protein level of at least about 2.5 g/L.

34. The method of claim 19, wherein said culturing is conducted in a reaction volume of at least about 15 L.

35. The method of claim 19, wherein said enzyme mixture comprises at least one enzyme selected from cellobiohydrolase, endoglucanase, beta-glucosidase, glycosyl hydrolase, xylanase, glucanase, pectinase, amylase, glucoamylase, lipase, protease, esterase, glucose isomerase, glucose oxidase, and phytase.

36. The method of claim 1, wherein said culturing medium comprises a feedstock.

37-39. (canceled)

40. A method for producing fermentable sugars comprising contacting the enzyme produced using the methods of claim 1, with at least one cellulosic feedstock under conditions whereby said fermentable sugars are produced.

41. A method for producing at least one end product from at least one cellulosic substrate, comprising: a) providing at least one cellulosic substrate and at least one enzyme composition comprising at least one enzyme or enzyme mixture produced according to claim 1; b) contacting said cellulosic substrate with the enzyme composition under conditions whereby fermentable sugars are produced from the cellulosic substrate in a saccharification reaction; and c) contacting the fermentable sugars with a microorganism under fermentation conditions such that at least one end product is produced.

42. The method of claim 41, wherein said method comprises simultaneous saccharification and fermentation reactions (SSF), or separate reactions (SHF).

43-50. (canceled)

51. The method of claim 41, wherein said microorganism is a yeast.

52. (canceled)

53. The method of claim 41, further comprising recovering at least one fermentation end product.
